(19) United States
(12) Patent Application Publication
(10) Pub. No.: US 2011/0274637 A1
(43) Pub. Date: Nov. 10, 2011

(54) Ethylene Oxide Free Antiperspirant/Deodorant Formulations

(75) Inventors: Jadranka Milardovic, Dusseldorf (DE); Petra Schulte, Koln (DE); Gabriele Strauss, Dusseldorf (DE); Ev Suess, Mannheim (DE)

(73) Assignee: Cognis IP Management GmbH, Dusseldorf (DE)

(21) Appl. No.: 13/131,443

(22) PCT Filed: Nov. 19, 2009
(86) PCT No.: PCT/EP2009/008231

§ 371 Date: May 26, 2011

(30) Foreign Application Priority Data
Nov. 28, 2008 (EP) 080207046

(51) Int. Cl. A61K 8/81; A61Q 15/00

(52) U.S. Cl. 424/65

(57) ABSTRACT

This invention relates to ethylene oxide free antiperspirant/deodorant formulations comprising at least one antiperspirant/deodorant active ingredient and at least one quaternary polymer having the INCI designation polyquaternium 37.

### FIELD OF THE INVENTION

The present invention relates to ethylene oxide free antiperspirant/deodorant preparations based on the quaternary cationic polymer with the INCI name Polyquaternium 37.

### PRIOR ART

Antiperspirant/deodorant formulations are known in the prior art. In need of improvement in the case of existing formulations is the long-time stability of the emulsions, the sensory effect while simultaneously retaining the antiperspirant/deodorant performance. There is therefore a continuing need for improved antiperspirant/deodorant formulations.

Antiperspirant/deodorant formulations on the market are generally ethylene oxide-containing since the emulsifiers used in the formulations usually comprise ethylene oxide (EO), which is regarded as critical from an ecological point of view, although they have an adequate performance as regards the protective effect against perspiration odor. There are also EO-free antiperspirant/deodorant formulations on the market which are generally based on glyceryl stearate citrate and are not convincing either in terms of the stability or in terms of their sensory performance since they are associated with considerable stickiness. Although there are solution approaches in the literature for achieving a dry end feel, these relate exclusively to anhydrous systems (W. Umbach, Kosmetik and Hygiene [Cosmetic and hygiene], Wiley-VCH, 3rd edition, p. 371), which do rightly have a dry skin feel after application, although this appears harsh and unpleasant after a short time.

It was an object of the present invention to provide antiperspirant/deodorant formulations which firstly have good stability and secondly absorb into the skin directly after application and which leave behind a dry, non-sticky end feel without the protective effect being reduced. Preferably, the antiperspirant/deodorant formulations should have a viscosity in the range from 900 to 15 000 mPas (Brookfield RVT; 23° C.; spindle 5; 10 rpm). It has been found that the preparations according to the invention achieve these objects.

### DESCRIPTION OF THE INVENTION

The present invention relates to ethylene oxide free antiperspirant/deodorant preparations with the following constituents:
a. at least one antiperspirant/deodorant active ingredient,
b. at least one quaternary polymer with the INCI name Polyquaternium 37.

In one preferred embodiment, the preparations comprise:
a. at least one antiperspirant/deodorant active ingredient,
b. at least one quaternary polymer with the INCI name Polyquaternium 37, and
c. a lipophilic phase which is formed from liquid oil and/or wax.

Preference is given to antiperspirant/deodorant preparations which comprise the constituents in the following fractions:
a. 1-30% by weight, preferably 5-20% by weight, and very particularly preferably 10-15% by weight, of the antiperspirant/deodorant active ingredient,
b. 0.1-10% by weight and preferably 1-5% by weight of a quaternary polymer with the structural formula specified below and the INCI name Polyquaternium 37, and
c. 1-20% by weight, preferably 5-15% by weight, of the lipophilic phase,
in each case based on the total weight of the antiperspirant/deodorant preparation.

The term “ethylene oxide free preparations” is understood as meaning that the preparations comprise no compounds which contain ethylene oxide units.

Polyquaternium 37 is a quaternary polymer which corresponds to the following general formula:

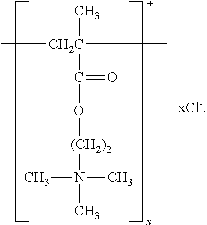

Surprisingly, it has been found that ethylene oxide free antiperspirant/deodorant preparations having improved stability can be produced if the preparations comprise the quaternary polymer with the INCI name Polyquaternium 37. Surprisingly, it has been found that ethylene oxide free antiperspirant/deodorant preparations can be produced with a viscosity in the range from 900 to 15 000 mPas, preferably with a viscosity of from 1500 to 10 000 mPas and very particularly preferably with a viscosity of 2000-5000 mPas (Brookfield RVT; 23° C.; spindle 5; 10 rpm). In one preferred embodiment, the preparations comprise no interface-active substance, in particular no emulsifier. In one preferred embodiment, the preparations comprise no interface-active substance, in particular no emulsifier, and comprise a lipophilic phase. Moreover, these preparations are characterized in that they additionally have significantly better sensory properties. In particular, with Polyquaternium 37, the sticky, harsh and waxy skin feel upon use, which is present in the case of prior art preparations, e.g. preparations which comprise glyceryl stearate citrate, can be avoided or at least significantly reduced. In spite of all this, the protective performance, i.e. the effect as antiperspirant/deodorant, is retained.

Polyquaternium 37 grades which are commercially available and can be used according to the invention are e.g.: Ultragel® 300—Cognis GmbH, OriStar PQ37—Orient Stars LLC, Synthalen CN—3V Group, Synthalen CR—3V Group, Synthalen CU—3V Group, Syntran PC 5320—Interpolymer Corporation; Cosmedia® Triple C (Cognis GmbH; INCI: Polyquaternium-37, Dicaprylyl Carbonate, Lauryl Glucoside).

In one embodiment of the invention, the Polyquaternium-37 is in the form of the commercially available formulation.

Preference is therefore given to an antiperspirant/deodorant preparation which comprises the Polyquaternium 37 generally in a fraction of 0.1-10% by weight, based on the total weight of the antiperspirant/deodorant preparation, ideally 1 to 5% by weight.

### Interface-Active Substance

The preparations according to the invention can comprise one or more interface-active substances. The compositions according to the invention comprise the interface-active substance(s) in an amount of from 0 to 80% by weight, in particular 0 to 40% by weight, preferably 0.1 to 20% by weight, preferably 0.1 to 15% by weight and in particular 0.1 to 10% by weight, based on the total weight of the composition.

Suitable interface-active substances are in principle any substance which lowers the surface tension between the aqueous phase and the nonaqueous phase. Interface-active substances include emulsifiers and surfactants.

A suitable emulsifier is in principle any interface-active substance, but in particular substances with an HLB value of from 1 to 20 according to the Griffin scale. Each emulsifier is ascribed a so-called HLB value (a dimensionless number between 1 and 20, Griffin scale), which indicates whether a preferred water solubility or oil solubility is present. Numbers below preferably characterize oil-soluble, hydrophobic emulsifiers, and numbers above 11 characterize water-soluble, hydrophilic emulsifiers. The HLB value says something about the balance between the size and strength of the hydrophilic and lipophilic groups in an emulsifier. The Griffin scale is described in WC Griffin, J. Soc. Cosmet. Chem. 1 (1949) 311; WC Griffin, J. Soc. Cosmet. Chem. 5 (1954) 249.

The HLB value of an emulsifier can also be calculated from increments, where the HLB increments for the different hydrophilic and hydrophobic groups from which a molecule is composed can be found in tabular works (e.g. H. P. Fiedler, Lexikon der Hilfsstoffe für Pharmazie, Kosmetik and angrenzende Gebiete [Lexicon of auxiliaries for pharmacy, cosmetic and related fields], Editio Cantor Verlag, Aulendorf, 4th edition 1996) or the manufacturer's data. The solubility of the emulsifier in the two phases practically determines the emulsion type. If the emulsifier is more soluble in water, this gives a O/W emulsion. On the other hand, if the emulsifier has better solubility in the oil phase, under otherwise identical preparation conditions, a W/O emulsion is formed.

In one preferred embodiment of the invention, the preparations comprise less than 1% by weight, in particular less than 0.5% by weight, preferably less than 0.1% by weight, of an interface-active substance, in particular less than 1% by weight, in particular less than 0.5% by weight, preferably less than 0.1% by weight, of an emulsifier. In one embodiment of the invention, the preparations comprise no surface-active substance, in particular no emulsifiers.

In one preferred embodiment of the invention, the preparations comprise a lipophilic phase and less than 1% by weight, in particular less than 0.5% by weight, preferably less than 0.1% by weight, of an interface-active substance. In one embodiment of the invention, the preparations comprise one lipophilic phase and no interface-active substance.

Usually, antiperspirant/deodorant formulations comprise ethylene oxide containing emulsifiers/interface-active substances. However, as mentioned at the start, ethylene oxide is ecologically unacceptable, and there is therefore the desire to avoid it in cosmetic preparations. According to the invention, no ethylene oxide containing substance is present in the antiperspirant/deodorant preparations. In one preferred embodiment, no emulsifier is present in the antiperspirant/deodorant preparations according to the invention. In another embodiment of the present invention, an emulsifier and optionally coemulsifier may be present in the antiperspirant/deodorant preparations provided these and the entire formulation comprise no ethylene oxide containing compounds. If such an emulsifier is present, it may be the emulsifiers customarily used in cosmetic preparations, which are also used in customary amounts. For examples of the emulsifiers and coemulsifiers which can be used, reference is made to WO 2008/019773, page 7, 5th paragraph to page 12, 2nd paragraph, with the proviso that they are not ethylene oxide containing emulsifiers and coemulsifiers.

In one embodiment of the invention, the preparations comprise at least one alkyl oligoglycoside as interface-active substance. C₈-C₂₂-Alkyl mono- and oligoglycosides, their preparation and their use are known from the prior art. Their preparation takes place in particular by reacting glucose or oligosaccharides with primary alcohols having 6 to 24, preferably 8 to 22, carbon atoms. As regards the glycoside radical, both monoglycosides, in which one cyclic sugar radical is glycosidically bonded to the fatty alcohol, and oligomeric glycosides with a degree of oligomerization up to preferably about 8 are suitable. The degree of oligomerization here is a statistical mean value based on a homolog distribution customary for said technical-grade products. Preferred alkyl oligoglycosides are compounds of the general formula Gₘ-R¹, in which G is a sugar radical having 5 or 6 carbon atoms, R¹ is a C6-C22 alkyl and/or alkenyl radical in acetal bond, and m is a mean value from 1 to 3. Lauryl glucoside, for example, can be used advantageously as alkyl oligoglycoside. Products which are available under the name Plantacare® or Plantaren® comprise a glucosidically bonded C₈-C₁₆-alkyl group on an oligoglucoside radical, the mean degree of oligomerization of which is 1 to 2. Acylglucamides derived from glucamine are also suitable as interface-active substances. According to the invention, preference is given to a product which is sold under the name Emulgade® PL 68/50 by Cognis Deutschland GmbH and is a 1:1 mixture of alkyl polyglucosides and fatty alcohols. According to the invention, it is also advantageously possible to use a mixture of lauryl glucoside, polyglyceryl-2 dipolyhydroxystearate, glycerol and water, which is commercially available under the name Eumulgin® VL 75.

### Antiperspirant/Deodorant Active Ingredient

According to the invention, suitable antiperspirant/deodorant active ingredients are all active ingredients which counteract body odors, conceal them or eliminate them. Body odors are produced as a result of the effect of skin bacteria on apokrin perspiration, whereupon unpleasant smelling degradation products are formed. Suitable antiperspirant/deodorant active ingredients are in particular compounds selected from the group consisting of antiperspirants, esterase inhibitors, bactericidal or bacteriostatic active ingredients and/or perspiration-absorbing substances.

The antiperspirant/deodorant active ingredient or the antiperspirant/deodorant active ingredients is/are present in customarily used amounts, preferably in a fraction of from 1 to 30% by weight, based on the total weight of the antiperspirant/deodorant preparation, more preferably 5 to 20% by weight and even more preferably 10 to 15% by weight. A single antiperspirant/deodorant active ingredient may be present, or a plurality of antiperspirant/deodorant active ingredients may be present. In the latter case, the percentages given above refer to the total amount of the antiperspirant/deodorant active ingredients present.

### Antiperspirants

Antiperspirants are salts of aluminum, zirconium or zinc. Such suitable antihydrotic active ingredients are, for example, aluminum chloride, aluminum chlorohydrate, aluminum dichlorohydrate, aluminum sesquichlorohydrate and complex compounds thereof, e.g. with 1,2-propylene glycol. Aluminum hydroxyallantoinate, aluminum chloride tartrate, aluminum zirconium trichlorohydrate, aluminum zirconium tetrachlorohydrate, aluminum zirconium pentachlorohydrate and complex compounds thereof e.g. with amino acid such as glycine. Preference is given to using aluminum chlorohydrate, aluminum zirconium tetrachlorohydrate, aluminum zirconium pentachlorohydrate and complex compounds thereof, such as e.g. Locron L or ACH-303 50% solution, Rezal 67 or AZ-7373 Powder, Rezal 36 GC or AZG-7226 50% solution.

The preparations according to the invention can comprise the antiperspirants in amounts of from 1 to 20, preferably 5 to 20 and in particular 8 to 20% by weight—based on the total weight of the antiperspirant/deodorant preparation.

### Esterase Inhibitors

In the presence of perspiration in the axillary area, extracellular enzymes—esterases, preferably proteases and/or lipases—are formed by bacteria; these cleave esters present in the perspiration and thereby release odorous substances. Suitable esterase inhibitors are preferably trialkyl citrates such as trimethyl citrate, tripropyl citrate, triisopropyl citrate, tributyl citrate and in particular triethyl citrate (Hydagen® CAT, Cognis GmbH, Dusseldorf/FRG). The substances inhibit the enzyme activity and thereby reduce the formation of odor. Further substances which are suitable as esterase inhibitors are sterol sulfates or phosphates, such as, for example, lanosterol, cholesterol, campesterol, stigmasterol and sitosterol sulfate and phosphate, dicarboxylic acids and esters thereof, such as, for example, glutaric acid, monoethyl glutarate, diethyl glutarate, adipic acid, monoethyl adipate, diethyl adipate, malonic acid and diethyl malonate, hydroxycarboxylic acids and esters thereof, such as, for example, citric acid, malic acid, tartaric acid or diethyl tartrate, and zinc glycinate.

The preparations according to the invention can comprise the esterase inhibitors in amounts of from 0.01 to 20, preferably 0.1 to 10 and in particular 0.3 to 5% by weight—based on the total weight of the antiperspirant/deodorant preparation.

Bactericidal and bacteriostatic active ingredients Typical examples of suitable bactericidal and bacteriostatic active ingredients are in particular chitosan and phenoxyethanol. 5-Chloro-2-(2,4-dichlorophenoxy)phenol has also proven particularly effective; this is sold under the trade name Irgasan® by Ciba-Geigy, Basle/CH. Suitable germicidal agents are in principle all substances that are effective against Gram-positive bacteria, such as, for example 4-hydroxybenzoic acid and its salts and esters, N-(4-chlorophenyl)-N′-(3,4-dichlorophenyl)urea, 2,4,4′-trichloro-2′-hydroxydiphenyl ether (triclosan), 4-chloro-3,5-dimethylphenol, 2,2′-methylenebis(6-bromo-4-chlorophenol), 3-methyl-4-(1-methylethyl)phenol, 2-benzyl-4-chlorophenol, 3-(4-chlorophenoxy)-1,2-propanediol, 3-iodo-2-propynyl butylcarbamate, chlorhexidine, 3,4,4′-trichlorocarbanilide (TTC), antibacterial fragrances, thymol, thyme oil, eugenol, clove oil, menthol, mint oil, farnesol, phenoxyethanol, glycerol monocaprate, glycerol monocaprylate, glycerol monolaurate (GML), diglycerol monocaprate (DMC), N-alkylamides of salicylic acid, such as e.g. N-n-octylsalicylamide or N-n-decylsalicylamide.

The preparations according to the invention can comprise the bactericidal or bacteriostatic active ingredients in amounts of from 0.01 to 5 and preferably 0.1 to 2% by weight—based on the total weight of the antiperspirant/deodorant preparation.

### Perspiration-Absorbing Substances

Suitable perspiration-absorbing substances are modified starch, such as e.g. Dry Flo Plus (National Starch), silicates, talc and other substances of similar modification which appear to be suitable for the absorption of perspiration. The preparations according to the invention can comprise the perspiration-absorbing substances in amounts of from 0.1 to 20, preferably 1 to 20 and in particular 2 to 8% by weight—based on the total weight of the antiperspirant/deodorant preparation.

### Lipophilic Phase

According to the invention, the lipophilic phase is formed from or consists of wax(es) and/or oils. Preference is given to a combination of oils and waxes. The lipophilic phase is preferably present in a fraction of 1-20% by weight, based on the total weight of the antiperspirant/deodorant preparation, ideally 5-15% by weight.

The term “oils” (used synonymously: oil component) is used to refer to water-insoluble organic compounds which are liquid at 30° C. and which have a relatively low vapor pressure. The common feature of the oils is not their corresponding chemical constitution, but their similar physical consistency.

Suitable oil components are, for example, the classes of compounds specified below, provided these are liquid at 30° C. Thus e.g. Guerbet alcohols based on fatty alcohols having 6 to 18, preferably 8 to 10, carbon atoms (e.g. Eutanol® G), esters of linear C₆-C₂₂-fatty acids with linear or branched C₆-C₂₂-fatty alcohols (e.g. Cetiol® Sensoft) and esters of branched C₆-C₁₃-carboxylic acids with linear or branched C₆-C₂₂-fatty alcohols, such as e.g. myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate, erucyl erucate and hexyldecylstearate (Eutanol® G 16 S). Also suitable are esters of linear C₆-C₂₂-fatty acids with branched alcohols, in particular 2-ethylhexanol, esters of C₃-C₃₈-alkylhydroxycarboxylic acids with linear or branched C₆-C₂₂-fatty alcohols—in particular dioctyl malate —, esters of linear and/or branched fatty acids with polyhydric alcohols (such as e.g. propylene glycol, dimerdiol or trimertriol) and/or Guerbet alcohols, triglycerides based on C₆-C₁₀-fatty acids, liquid mono-/di-/triglyceride mixtures based on C₆-C₁₈-fatty acids, esters of C₆-C₂₂-fatty alcohols and/or Guerbet alcohols with aromatic carboxylic acids, in particular benzoic acid, esters of C₂-C₁₂-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, such as e.g. 1,3-dialkylcyclohexanes, linear and branched C₆-C₂₂-fatty alcohol carbonates, such as e.g. dicaprylyl carbonate (Cetiol® CC), Guerbet carbonates based on fatty alcohols having 6 to 18, preferably 8 to 10, carbon atoms, esters of benzoic acid with linear and/or branched C₆-C₂₂-alcohols (e.g. Finsolv® TN), linear or branched, symmetrical or asymmetrical dialkyl ethers having 6 to 22 carbon atoms per alkyl group, such as e.g. dicaprylyl ether (Cetiol® OE), ring-opening products of epoxidized fatty acid esters with polyols (Hydagen® HSP, Sovermol® 750, Sovermol® 1102), silicone oils, (cyclomethicones, silicon methicone grades etc. and/or aliphatic and naphthenic hydrocarbons, such as e.g. mineral oil, Vaseline, petrolatum, squalane, squalene or dialkylcyclohexanes.

Suitable further oil bodies are, for example, silicone oils. They can be present as cyclic and/or linear silicone oils. Silicone oils are high molecular weight synthetic polymeric compounds in which silicon atoms are linked via oxygen atoms in a chain-like and/or reticular manner and the remaining valences of the silicon are saturated by hydrocarbon radicals (in most cases methyl groups, less frequently ethyl, propyl, phenyl groups etc.). Systematically, the silicone oils are referred to as polyorganosiloxanes. The methyl-substituted polyorganosiloxanes, which are the most important compounds of this group in terms of amount and are characterized by the following structural formula

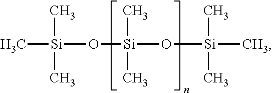

are also referred to as polydimethylsiloxane or dimethicone (INCI). Dimethicones come in various chain lengths and with various molecular weights. Advantageous polyorganosiloxanes within the context of the present invention are, for example, dimethylpolysiloxane [poly(dimethylsiloxane)], which are available for example under the trade names Abil 10 to 10 000 from Evonik Goldschmidt. Also advantageous are phenylmethylpolysiloxane (INCI: Phenyl Dimethicone, Phenyl Trimethicone), cyclic silicones (octamethylcyclotetrasiloxane and decamethylcyclopentasiloxane), which are also referred to in accordance with INCI as Cyclomethicone, amino-modified silicones (INCI: Amodimethicones) and silicone waxes, e.g. polysiloxanepolyalkylene copolymers (INCI: Stearyl Dimethicone and Cetyl Dimethicone) and dialkoxydimethylpolysiloxanes (Stearoxy Dimethicone and Behenoxy Stearyl Dimethicone), which are available as various Abil wax grades from Evonik Goldschmidt. However, other silicone oils can also be used advantageously within the context of the present invention, for example cetyl dimethicone, hexamethylcyclotrisiloxane, polydimethylsiloxane, poly(methylphenylsiloxane). Silicones that are particularly preferred according to the invention are dimethicone and cyclomethicone.

Suitable oil components are also polycarbonates, as described for example in WO 03/041676, to which reference is expressly made here. The particularly suitable polycarbonate is that under the INCI name Hydrogenated Dimer Dilinoleyl/Dimethylcarbonate Copolymer, which is available as commercial product Cosmedia® DC from Cognis GmbH.

The dialkyl carbonates and dialkyl ethers may be symmetrical or asymmetrical, branched or unbranched, saturated or unsaturated and can be prepared by reactions which are sufficiently known from the prior art.

According to the invention, it is also possible to use, inter alia, hydrocarbons, preferably having a chain length of 8 to 40 carbon atoms. They may be branched or unbranched, saturated or unsaturated. Among these, preference is given to branched, saturated C₈-C₄₀-alkanes. It is possible to use either pure substances or substance mixtures. They are usually substance mixtures of different isomeric compounds. Compositions which have alkanes with 10 to 30, preferably 12 to 20, and particularly preferably 16 to 20 carbon atoms are particularly suitable, and, among these, a mixture of alkanes which comprises at least 10% by weight of branched alkanes based on the total amount of the alkanes. They are preferably branched, saturated alkanes. Mixtures of alkanes which comprise more than 1% by weight of 5,8-diethyldodecane and/or more than 1% by weight of didecene are particularly suitable. Also suitable are esters of 2-propylheptanol with n-octanoic acid, as e.g. commercially available under the trade name Cetiol® SenSoft (Cognis GmbH). Also suitable are hydrocarbons, such as for example undecane and tridecane. Also suitable are alkanes, such as e.g. the mixtures with the INCI name Coconut/Palm/Palm Kernel Oil Alkanes (trade name Vegelight 1214 from Biosynthesis).

According to the invention, it is possible to use an oil or a mixture of two or more oil components.

The term wax (used synonymously: wax component) is usually understood as meaning all natural or artificially obtained substances and substance mixtures having the following properties: they are of solid to brittly hard consistency, coarse to finely divided, transparent to cloudy and melt above 30° C. without decomposition. They are of low viscosity even a little above the melting point and are not thread-drawing and exhibit a strongly temperature-dependent consistency and solubility. According to the invention, it is possible to use one wax component or a mixture of wax components which melt at 30° c. or above. Waxes which can be used according to the invention are also fats and fat-like substances with wax-like consistency, provided they have the required melting point. These include, inter alia, fats (triglycerides), and natural and synthetic waxes or any desired mixtures of these substances. Fats are understood as meaning triacylglycerols, i.e. the triple esters of fatty acids with glycerol. Preferably, they comprise saturated, unbranched and unsubstituted fatty acid radicals. These may also be mixed esters, i.e. triple esters of glycerol with various fatty acids. According to the invention, so-called hydrogenated fats and oils, which are obtained by partial hydrogenation, can also be used. Vegetable hydrogenated fats and oils are preferred, e.g. hydrogenated castor oil, peanut oil, soybean oil, colza oil, rapeseed oil, cottonseed oil, soybean oil, sunflower oil, palm oil, palm kernel oil, linseed oil, almond oil, corn oil, olive oil, sesame oil, cocoa butter and coconut fat. Inter alia, the triple esters of glycerol with C₁₂-C₆₀-fatty acids and in particular C₁₂-C₃₆-fatty acids are suitable. These include hydrogenated castor oil, a triple ester of glycerol and a hydroxy stearic acid, which is commercially available for example under the name Cutina® HR. Glycerol tristearate, glycerol tribehenate (e.g. Syncrowax® HRC), glycerol tripalmitate or the triglyceride mixtures known under the name Syncrowax®HGLC are likewise suitable, with the proviso that the melting point of the wax component or of the mixture is 30° C. or above.

According to the invention, wax components which can be used are in particular mono- and diglycerides and mixtures of these partial glycerides. Glyceride mixtures which can be used according to the invention include the products Novata AB and Novata B (mixture of C₁₂-C₁₈-mono-, di- and triglycerides) and Cutina MD or Cutina GMS (glyceryl stearate) marketed by Cognis GmbH.

Further wax components are from the group of the C₆-C₂₂-fatty acid esters of pentaerythritol, of dipentaerythritol, of tripentaerythritol or of any desired mixture of these esters which have a melting point of at least 30° C. One preferred pentaerythritol ester mixture consists of a fraction of 5-35% by weight of monoesters, 20-50% by weight of diesters and 25-50% by weight of triesters, and optionally tetraesters. Particular preference is given to a content of 10-25% by weight of monoesters, 25-40% by weight of diesters and 30-45% by weight of triesters, and optionally tetraesters and very particularly preferably 12-19% by weight of monoesters, 25-35% by weight of diesters and 30-40% by weight of triesters and 6-11% by weight of tetraesters. The pentaerythritol ester mixture which can be used according to the invention includes the product Cutina® PES marketed by Cognis GmbH.

Fatty alcohols which can be used according to the invention as wax component include the C₁₂-C₅₀-fatty alcohols. The fatty alcohols can be obtained from natural fats, oils and waxes, such as, for example, myristyl alcohol, 1-pentadecanol, cetyl alcohol, 1-heptadecanol, stearyl alcohol, 1-nonadecanol, arachidyl alcohol, 1-heneicosanol, behenyl alcohol, brassidyl alcohol, lignoceryl alcohol, ceryl alcohol or myricyl alcohol. According to the invention, preference is given to saturated unbranched fatty alcohols. However, unsaturated, branched or unbranched fatty alcohols can also be used according to the invention as wax component provided they have the required melting point. According to the invention, it is also possible to use fatty alcohol cuts, as are produced during the reduction of naturally occurring fats and oils, such as e.g. bovine tallow, peanut oil, colza oil, cottonseed oil, soybean oil, sunflower oil, palm kernel oil, linseed oil, castor oil, corn oil, rapeseed oil, sesame oil, cocoa butter and coconut fat. However, it is also possible to use synthetic alcohols, e.g. the linear, even-numbered fatty alcohols of the Ziegler synthesis (alfols) or the partially branched alcohols from the oxo synthesis (dobanols). According to the invention, C₁₄-C₂₂-fatty alcohols, which are marketed for example by Cognis GmbH under the name Lanette 18 (C₁₈-alcohol), Lanette 16 (C₁₆-alcohol), Lanette 14 (C₁₄-alcohol), Lanette 0 (C₁₆/C₁₈-alcohol) and Lanette 22 (C₁₈/C₂₂-alcohol), are particularly preferably suitable. Fatty alcohols give the preparations a dryer skin feel than triglycerides and are therefore preferred over the latter.

Wax components which can be used are also C₁₄-C₄₀-fatty acids or mixtures thereof. These include, for example, myristic acid, pentadecanoic acid, palmitic acid, margaric acid, stearic acid, nonadecanoic acid, arachic acid, behenic aid, lignoceric acid, cerotic acid, melissic acid, erucic acid and eleostearic acid, and also substituted fatty acids, such as e.g. 12-hydroxystearic acid, and the amides or monethanolamides of the fatty acids, this list being exemplary and nonlimiting in character.

According to the invention, it is possible to use, for example, natural vegetable waxes, such as candelilla wax, carnauba wax, Japan wax, espartograss wax, cork wax, guaruma wax, rice germ oil wax, sugar cane wax, ouricuryi wax, montan wax, sunflower wax, fruit waxes such as orange waxes, lemon waxes, grapefruit wax, bayberry wax, and animal waxes, such as e.g. beeswax, shellac wax, spermaceti, wool wax and uropygial grease. Within the context of the invention, it may be advantageous to use hydrogenated or hardened waxes. Natural waxes that can be used according to the invention also include the mineral waxes, such as e.g. ceresin and ozokerite, or the petrochemical waxes, such as e.g. petrolatum, paraffin waxes and microwaxes. Wax components which can be used are also chemically modified waxes, in particular the hard waxes, such as e.g. montan ester waxes, sasol waxes and hydrogenated jojoba waxes. Synthetic waxes which can be used according to the invention include, for example, wax-like polyalkylene waxes and polyethylene glycol waxes. Vegetable waxes are preferred according to the invention.

The wax component can likewise be selected from the group of wax esters of saturated and/or unsaturated, branched and/or unbranched alkanecarboxylic acids and saturated and/or unsaturated, branched and/or unbranched alcohols, from the group of esters of aromatic carboxylic acids, dicarboxylic acids, tricarboxylic acids and hydroxycarboxylic acids (e.g. 12-hydroxystearic acid) and saturated and/or unsaturated, branched and/or unbranched alcohols, and also from the group of lactides of long-chain hydroxycarboxylic acids. Example of such esters are the C₁₆-C₄₀-alkyl stearates, C₂₀-C₄₀-alkyl stearates (e.g. kester wax K82H), C₂₀-C₄₀-dialkyl esters of dimeric acids, C₁₈-C₃₈-alkylhydroxystearoyl stearates or C₂₀-C₄₀-alkyl erucates. C₃₀-C₅₀-Alkyl beeswax, tristearyl citrate, triisostearyl citrate, stearyl heptanoate, stearyl octanoate, trilauryl citrate, ethylene glycol dipalmitate, ethylene glycol distearate, ethylene glycol di(12-hydroxystearate), stearyl stearate, palmityl stearate, stearyl behenate, cetyl ester, cetearyl behenate and behenyl behenate can also be used. Fatty acid partial glycerides, i.e. technical-grade mono- and/or diesters of glycerol with fatty acids having 12 to 18 carbon atoms, such as, for example, glyceryl mono/dilaurate, -palmitate, myristate or -stearate, are also suitable for this purpose.

Suitable waxes are also pearlescent waxes. Suitable pearlescent waxes, especially for use in surface-active formulations, are, for example: alkylene glycol esters, specifically ethylene glycol distearate; fatty acid alkanolamides, specifically coconut fatty acid diethanolamide; partial glycerides, specifically stearic acid monoglyceride; esters of polybasic, optionally hydroxy-substituted carboxylic acids with fatty alcohols having 6 to 22 carbon atoms, specifically long-chain esters of tartaric acid; fatty substances, such as for example fatty alcohols, fatty ketones, fatty aldehydes, fatty ethers and fatty carbonates, which have in total at least 24 carbon atoms, specifically laurone and distearyl ethers; fatty acids such as stearic acid, hydroxy stearic acid or behenic acid, ring-opening products of olefin epoxides having 12 to 22 carbon atoms with fatty alcohols having to 22 carbon atoms and/or polyols having 2 to 15 carbon atoms and 2 to 10 hydroxyl groups, and mixtures thereof.

According to the invention, it is possible to use a wax or a mixture of two or more waxes.

### Surfactants

In one embodiment of the invention, the preparations according to the invention comprise at least one surfactant as interface-active substance. Surfactants are amphiphilic substances which can dissolve organic, nonpolar substances in water. As a result of their specific molecular structure with at least one hydrophilic and one hydrophobic molecular moiety, they provide for a reduction in the surface tension of water, wetting of the skin, facilitation of soil removal and dissolution, ease of rinsing off and—if desired—for foam regulation. Surfactants are usually understood as meaning interface-active substances which have an HLB value of greater than 20.

Interface-active substances which may be present are anionic, nonionic, cationic and/or amphoteric or zwitterionic surfactants. In surfactant-containing cosmetic preparations, preferably at least one anionic surfactant is present.

Typical examples of nonionic surfactants are mixed ethers and mixed formals, optionally partially oxidized alk(en)yl oligoglycosides and glucoronic acid derivatives, fatty acid N-alkylglucamides, protein hydrolysates (in particular wheat-based vegetable products), polyol fatty acid esters, sugar esters, sorbitan esters, polysorbates and amine oxides. Zwitterionic surfactants is the term used to refer to those surface-active compounds which carry at least one quaternary ammonium group and at least one—COO⁽⁻⁾ or—SO₃⁽⁻⁾ group in the molecule. Particularly suitable zwitterionic surfactants are the so-called betaines, such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazoline having in each case 8 to 18 carbon atoms in the alkyl or acyl group, and also cocoacylaminoethyl hydroxyethylcarboxymethyl glycinate. A preferred zwitterionic surfactant is the fatty acid amide derivative known under the INCI name Cocamidopropyl Betaine. Likewise suitable, especially as cosurfactants, are ampholytic surfactants. Ampholytic surfactants are understood as meaning those surface-active compounds which, apart from a C₈-C₁₈-alkyl or acyl group in the molecule, contain at least one free amino group and at least one—COOH or—SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylaminopropylglycines, N-alkyltaurines, N-alkylsarcosines, 2-alkylaminopropionic acids and alkylaminoacetic acids having in each case about 8 to 18 carbon atoms in the alkyl group. Particularly preferred ampholytic surfactants are N-cocoalkylaminopropionate, cocoacylaminoethylaminopropionate and C₁₂₋₁₈-acylsarcosine. Typical examples of amphoteric and zwitterionic surfactants are alkylbetaines, alkylamidobetaines, aminopropionates, aminoglycinates, imidazolinium betaines and sulfobetaines. The specified surfactants are exclusively known compounds. With regard to structure and preparation of these substances, reference may be made to the relevant review works in this field. Typical examples of particularly suitable mild, i.e. particularly skin-compatible, surfactants are monoglyceride sulfates, mono- and/or dialkyl sulfosuccinates, fatty acid isethionates, fatty acid sarcosinates, fatty acid taurides, fatty acid glutamates, α-olefinsulfonates, ether carboxylic acids, alkyl oligoglucosides and/or mixtures thereof with alkyl oligoglucoside carboxylates, fatty acid glucamides, alkylamidobetaines, amphoacetals and/or protein fatty acid condensates, the latter preferably based on wheat proteins or salts thereof. Anionic surfactants are characterized by a water-solubilizing, anionic group such as e.g. a carboxylate, sulfate, sulfonate or phosphate group and a lipophilic radical. Skincompatible anionic surfactants are known to the person skilled in the art in a large number from relevant handbooks and are commercially available. These are in particular alkylsulfates in the form of their alkali metal, ammonium or alkanolammonium salts, alkyl ether sulfates, alkyl ether carboxylates, acyl isethionates, acyl sarcosinates, acyltaurines with liner alkyl or acyl groups having 12 to 18 carbon atoms, and sulfosuccinates and acyl glutamates in the form of their alkali metal or ammonium salts. Typical examples of anionic surfactants are soaps, alkylbenzenesulfonates, alkanesulfonates, olefinsulfonates, alkyl ether sulfonates, glyceryl ether sulfonates, α-methyl ester sulfonates, sulfo fatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids, such as, for example, acyl lactylates, acyl tartrates, acyl glutamates and acyl aspartates, alkyl oligoglucoside sulfates, protein fatty acid condensates (in particular vegetable products based on wheat) and alkyl (ether) phosphates. Cationic surfactants which can be used are in particular quaternary ammonium compounds. Preference is given to ammonium halides, in particular chlorides and bromides, such as alkyltrimethylammonium chlorides, dialkyldimethylammonium chlorides and trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride and tricetylmethylammonium chloride. Furthermore, the very readily biodegradable quaternary ester compounds, such as for example the dialkylammonium methosulfates and methylhydroxyalkyldialkoyloxyalkylammonium ethosulfates sold under the trade name Stepantex® and the corresponding products of the Dehyquart® series can also be used as cationic surfactants. The term “ester quats” is generally understood as meaning quaternized fatty acid triethanolamine ester salts. These are known substances which are prepared by the relevant methods of organic chemistry. Further cationic surfactants which can be used according to the invention are the quaternized protein hydrolysates.

Besides the specified constituents, other constituents customary in cosmetic preparations and in particular antiperspirant/deodorant formulations may be present in customary fractions. These include e.g. preservatives, biogenic active ingredients, thickeners, superfatting agents, stabilizers, polymers, antioxidants, film formers, swelling agent, insect repellent hydrotropes, solubilizers, perfume oils, dyes, pigments, UV filters etc.

The antiperspirant/deodorant preparations according to the invention usually comprise the further constituents in amounts of in total <25% by weight, in particular <20% by weight, based on the total weight of the antiperspirant/deodorant preparation.

Suitable preservatives are for example phenoxyethanol, formaldehyde solution, parabens, mixtures of phenoxyethanol and ethylhexylglyceryl (as are available for example under the trade name Euxyl PE 9010) or sorbic acid, and also the silver complexes known under the name Surfacine® and the other substance classes listed in Annex 6, Part A and B of the Cosmetic Ordinance. In a preferred embodiment of the invention, the preservative is selected from the group consisting of phenoxyethanol, formaldehyde solution, parabens, organic acids and mixtures thereof, optionally in combination with pentanediol and/or ethylhexylglycerol.

In one embodiment of the invention, the preparations according to the invention comprise at least one biogenic active ingredient as further constituent. Biogenic active ingredients are to be understood as meaning for example tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, such as e.g. prunus extract, bambara nut extract and vitamin complexes. In one preferred embodiment of the invention, the preparations according to the invention comprise at least one compound selected from vitamins, allantoin, bisabolol and plant extracts as biogenic active ingredient. In one preferred embodiment of the invention, the preparations according to the invention comprise at least one compound selected from tocopherol, tocopherol acetate, tocopherol palmitate, ascorbic acid (deoxy)ribonucleic acids and fragmentation products thereof, β-glucans, retinol, bisabolol, allantoin, phytantriol, panthenol, AHA acids, amino acids, ceramides, pseudoceramides, essential oils, plant extracts, such as e.g. Aloe Vera, prunus extract, bambara nut extract and vitamin complexes and mixtures thereof as biogenic active ingredient.

In one embodiment of the invention, the antiperspirant/deodorant preparations according to the invention comprise at least one thickener as further constituent.

Suitable thickeners are, for example, Aerosil grades (hydrophilic silicas), polysaccharides, in particular xanthan gum, guar guar, agar agar, alginates and tyloses, carboxymethylcellulose and hydroxyethyl- and hydroxypropylcellulose, polyvinyl alcohol, polyvinylpyrrolidone and bentonites, such as e.g. Bentone® gel VS-5PC (Rheox).

Suitable insect repellants are for example, N,N-diethyl-m-toluamide, 1,2-pentanediol or ethyl 3-(N-n-butyl-N-acetylamino)propionate, which is sold under the name Insect Repellent® 3535 by Merck KGaA, and also butylacetylaminopropionates.

Perfume oils which may be mentioned are mixtures of natural and synthetic fragrances. Natural fragrances are extracts from flowers, stems and leaves, fruits, fruit peels, roots, woods, herbs and grasses, needles and branches, resins and balsams. Also suitable are animal raw materials, such as for example civet and castoreum, and also synthetic fragrance compounds of the ester, ether, aldehyde, ketone, alcohol and hydrocarbon types.

The term pigment covers particles of any shape, which are white or colored, organic or inorganic, are insoluble in the preparations, and serve the purpose of coloring the preparation. In one preferred embodiment, inorganic pigments are used, particular preference being given to metal oxides.

Examples of inorganic pigments which may be mentioned are: titanium dioxide, optionally surface-coated, zirconium or cerium oxides and zinc, iron (black, yellow or red) and chromium oxides, manganese violet, ultramarine blue, chromium hydrate and iron(III) blue, metal powders such as aluminum powder or copper powder. In one preferred embodiment of the invention, the pigment is selected from the inorganic pigments, preferably from the metal oxides. In one preferred embodiment, the pigment is selected from the group consisting of titanium dioxide, zinc oxide, iron oxide and mixtures thereof. The pigments can be present either individually or else in mixtures. Within the context of the present invention, preference is given to pigment mixtures composed of white pigments (e.g. kaolin, titanium dioxide or zinc oxide) and inorganic colored pigments (e.g. iron oxide pigments, chromium oxides), where the pigments may be present in coated or uncoated form. Among the colored pigments, iron oxides are particularly preferred. Within the context of the present invention, the pigment(s) may also advantageously be selected from the group of the effect pigments, which impart to the cosmetic preparation, as well as the pure color, an additional property—such as e.g. angular dependence of the color (flop), luster (not surface luster) or texture. According to the invention, such effect pigments are used advantageously in addition to one or more white pigments and/or colored pigments.

The most important group of effect pigments is that of the luster pigments, which, according to DIN 55944: 2003-11, include the metal effect pigments and the pearlescent pigments. Some specific effect pigments cannot be assigned to these two groups, e.g. graphite platelets, iron oxide platelets and micronized titanium dioxide, where micronized titanium dioxide does not give a luster effect, but an angle-dependent light-scattering effect. The luster pigments according to DIN 55943: 2001-10 are predominantly effect pigment platelets. Oriented in parallel, luster pigments exhibit a characteristic luster. The visual effect of luster pigments is based on the directed reflection on metallic particles (metal effect pigments), on transparent particles with a high refractive index (pearlescent pigments) or on the phenomenon of interference (interference pigments) (DIN 55944: 2003-11). Examples of standard commercial effect pigments preferred according to the invention are: Timiron and #174; from Merck, Iriodin and #174; from Merck (pearlescent and color luster pigments for decorative technical applications), Xirallic and #174; from Merck (color-intense crystal effect pigments).

In addition, the preparations according to the invention can also advantageously comprise organic colored pigments, i.e. organic dyes which are practically insoluble in the preparation. According to DIN 55944: 1990-04, organic pigments can be divided according to chemical aspects into azo pigments and polycyclic pigments, and also according to color aspects into colored or black pigments. Organic white pigments are of no practical significance. Within the context of the present invention, the pigments may advantageously also be used in the form of commercially available oily or aqueous predispersions. The preparations according to the invention can comprise for example 0.1 to 40% by weight of pigments—based on the total weight of the cosmetic and/or pharmaceutical preparation.

It is also possible that the preparation according to the invention comprises one or more dyes. The dyes may be either of synthetic or natural origin. A list of suitable dyes can be found in EP 1 371 359 A2, p. 8, lines 25-57, p. 9 and p. 10, and also p. 11, lines 1 to 54, to which reference is hereby explicitly made. The preparations according to the invention usually comprise 0.01 to 5, preferably 0.1 to 1.0, % by weight of dyes—based on the total weight of the cosmetic and/or pharmaceutical preparation. The preparations according to the invention can comprise for example a total amount of dyes and pigments in the range from 0.01 to 30% by weight, in particular 0.1 to 15% by weight, preferably 1 to 10% by weight, based on the total weight of the cosmetic and/or pharmaceutical preparation. Suitable dyes and pigments are in particular the dyes and pigments approved according to Annex IV of the Commission Directive (in the version: Commission Directive 2007/22/EC of 17 Apr. 2007 amending Council Directive 76/768/EEC, concerning cosmetic products, for the purposes of adapting Annexes IV and VI thereto to technical progress), to which reference is hereby explicitly made.

According to the invention, suitable UV photoprotective filters are organic substances (photoprotective filters) that are crystalline or liquid at room temperature and which are able to absorb ultraviolet rays and release the absorbed energy again in the form of longer-wave radiation, e.g. heat. UV filters may be oil-soluble or water-soluble. Typical oil-soluble UV-B filters or broad-spectrum UV A/B filters to be mentioned are, for example:
3-benzylidenecamphor or 3-benzylidenenorcamphor (Mexoryl SDS 20) and derivatives thereof, e.g.
3-(4-methylbenzylidene)camphor as described in EP 0693471 B1
3-(4′-trimethylammonium)benzylidenebornan-2-one methyl sulfate (Mexoryl SO)
3,3′-(1,4-phenylenedimethine)bis(7,7-dimethyl-2-oxobicyclo[2.2.1]heptane-1-methanesulfonic acid) and salts (Mexoryl SX)
3-(4′-sulfo)benzylidenebornan-2-one and salts (Mexoryl SL)
polymer of N-{(2 and 4)-[2-oxoborn-3-ylidene)methyl}benzyl]acrylamide (Mexoryl SW)
2-(2H-benzotriazol-2-yl)-4-methyl-6-(2-methyl-3-(1,3,3,3-tetramethyl-1-(trimethylsilyloxy)disiloxanyl)propyl)phenol (Mexoryl SL)
4-aminobenzoic acid derivatives, preferably 2-ethylhexyl 4-(dimethylamino)benzoate, 2-octyl
4-(dimethylamino)benzoate and amyl 4-(dimethylamino) benzoate;
esters of cinnamic acid, preferably 2-ethylhexyl 4-methoxycinnamate, propyl 4-methoxycinnamate, isoamyl 4-methoxycinnamate, 2-ethylhexyl 2-cyano-3,3-phenylcinnamate (octocrylene);
esters of salicylic acid, preferably 2-ethylhexyl salicylate, 4-isopropylbenzyl salicylate, homomenthyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di-2-ethylhexyl 4-methoxybenzmalonate;
triazine derivatives, such as e.g. 2,4,6-trianilino(p-carbo-2′-ethyl-1′-hexyloxy)-1,3,5-triazine and 2,4,6-tris[p-(2-ethylhexyloxycarbonyl)anilino]-1,3,5-triazine (Uvinul T 150) as described in EP 0818450 A1 or bis(2-ethylhexyl) 4,4′-[(6-[4-((1,1-dimethylethyl)aminocarbonyl)phenylamino]-1,3,5-triazine-2,4-diyl)diimino]bisbenzoate (Uvasorb® HEB);
2,2-(methylenebis(6-(2H-benzotriazol-2-yl)-4-(1,1,3,3-tetramethylbutyl)phenol) (Tinosorb M);
2,4-bis[4-(2-ethylhexyloxy)-2-hydroxyphenyl]-6-(4-methoxyphenyl)-1,3,5-triazine (Tinosorb S);
propane-1,3-diones, such as e.g. 1-(4-tert-butylphenyl)-3-(4′-methoxyphenyl)propane-1,3-dione;
ketotricyclo(5.2.1.0)decane derivatives, as described in EP 0694521 B1;
dimethicodiethyl benzalmalonates (Parsol SLX).

Suitable water-soluble UV filters are:
2-phenylbenzimidazole-5-sulfonic acid and the alkali metal, alkaline earth metal, ammonium, alkylammonium, alkanolammonium and glucammonium salts thereof;
2,2-((1,4-phenylene)bis(1H-benzimidazole-4,6-disulfonic acid, monosodium salt) (Neo Heliopan AP)
sulfonic acid derivatives of benzophenones, preferably 2-hydroxy-4-methoxybenzophenone-5-sulfonic acid and its salts;
sulfonic acid derivatives of 3-benzylidenecamphor, such as e.g. 4-(2-oxo-3-bornylidenemethyl)benzenesulfonic acid and 2-methyl-5-(2-oxo-3-bornylidene)sulfonic acid and salts thereof.

Suitable typical UV-A filters are in particular derivatives of benzoylmethane, such as for example 1-(4′-tert-butylphenyl)-3-(4′-methoxyphenyl)propane-1,3-dione, 4-tert-butyl-4′-methoxydibenzoylmethane (Parsol® 1789), 1-phenyl-3-(4′-isopropylphenyl)propane-1,3-dione, and enamine compounds, as described in DE 19712033 A1 (BASF) and benzoic acid, 2-[4-(diethylamino)-2-hydroxybenzoyl], hexyl ester (Uvinal® A plus).

The UV-A and UV-B filters can of course also be used in mixtures. Particularly favorable combinations consist of the derivatives of benzoylmethane, e.g. 4-tert-butyl-4′-methoxydibenzoylmethane (Parsol® 1789) and 2-ethylhexyl 2-cyano-3,3-phenylcinnamate (octocrylene) in combination with esters of cinnamic acid, preferably 2-ethylhexyl 4-methoxycinnamate and/or propyl 4-methoxycinnamate and/or isoamyl 4-methoxycinnamate. Combinations of this type are advantageously combined with water-soluble filters such as e.g. 2-phenylbenzimidazole-5-sulfonic acid and alkali metal, alkaline earth metal, ammonium, alkylammonium, alkanolammonium and glucammonium salts thereof.

Suitable UV photoprotective filters are in particular the substances approved according to Annex VII of the Commission Directive (in the version Commission Directive 2005/9/EC of 28 Jan. 2005 amending Council Directive 76/768/EEC, concerning cosmetic products, for the purposes of adapting Annexes VII thereof to technical progress), to which reference is hereby explicitly made.

Besides the specified soluble substances, insoluble photoprotective pigments are also suitable for this purpose, namely finely disperse metal oxides and salts. Examples of suitable metal oxides are, in particular, zinc oxide and titanium dioxide and also oxides of iron, zirconium, silicon, manganese, aluminum and cerium, and mixtures thereof. Salts which can be used are silicates (talc), barium sulfate or zinc stearate. The oxides and salts are used in the form of the pigments for skincare and skin-protecting emulsions and also for decorative cosmetics. The particles should have an average diameter of less than 100 nm, preferably between 5 and 50 nm and in particular between 15 and 30 nm. They can have a spherical shape, although it is also possible to use those particles which have an ellipsoidal shape or a shape which deviates in some other way from the spherical configuration. The pigments can also be present in surface-treated form, i.e. hydrophilized or hydrophobized. Typical examples are coated titanium dioxides, such as e.g. titanium dioxide T 805 (Degussa) or Eusolex® T, Eusolex® T-2000, Eusolex® T-Aqua, Eusolex® AVO, Eusolex® T-ECO, Eusolex® T-OLEO and Eusolex® T-S (Merck). Typical examples are zinc oxides, such as e.g. zinc oxide neutral, zinc oxide NDM (Symrise) or Z-Cote® (BASF) or SUNZnO-AS and SUNZnO-NAS (Sunjun Chemical Co. Ltd.). Suitable hydrophobic coatings here are primarily silicones and specifically trialkoxyoctylsilanes or simethicones. In sunscreen compositions, preference is given to using so-called micropigments or nanopigments. Preference is given to using micronized zinc oxide. Further suitable UV photoprotective filters can be found in the review by P. Finkel in SÖFW-Journal 122, 8/1996, pp. 543-548 and Parf. 80^{th} volume, No. 3/1999, p. 10 to 16.

Besides the two aforementioned groups of primary photoprotective substances, it is also possible to use secondary photoprotective agents of the antioxidant type, which interrupt the photochemical reaction chain which is triggered when UV radiation penetrates into the skin. Typical examples thereof are amino acids (e.g. glycine, histidine, tyrosine, tryptophan) and derivatives thereof, imidazoles (e.g. urocaninic acid) and derivatives thereof, peptides such as D,L-carnosine, D-carnosine, L-carnosine and derivatives thereof (e.g. anserine), carotenoids, carotenes (e.g. -carotene, -carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, linoleyl, cholesteryl and glyceryl esters thereof), and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to mol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. gamma-linolenic acid, linolic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaicic acid, nordihydroguaiaretic acid, trihydroxybutyrophenone, uric acid and derivatives thereof, mannose and derivatives thereof, superoxide dismutase, zinc and derivatives thereof (e.g. ZnO, ZnSO4), selenium and derivatives thereof (e.g. selenomethionine), stilbene and derivatives thereof. (e.g. stilbene oxide, trans-stilbene oxide) and the derivatives (salts, esters, ethers, sugars, nucleotides, nucleosides, peptides and lipids) suitable according to the invention of these specified active ingredients.

Stabilizers which can be used are metal salts of fatty acids, such as e.g. magnesium, aluminum and/or zinc stearate and ricinoleate.

To improve the flow behavior, hydrotropes, such as for example ethanol, isopropyl alcohol, or polyols can be sued. Polyols which are suitable here preferably have 2 to 15 carbon atoms and at least two hydroxyl groups. The polyols can also contain further functional groups, in particular amino groups, and/or be modified with nitrogen.

The further constituents of the preparations (such as e.g. preservatives, cosmetic active ingredients, UV filters etc.) are added either via the water phase or via the lipophilic phase, depending on their solubility.

### Aqueous Fraction:

The preparations according to the invention can usually comprise 40 to 85, more preferably 50 to 75% by weight, of aqueous fraction, based on the total weight of the preparation.

The preparations according to the invention are generally present as emulsions, as a rule in the form of gels or gel creams (nontransparent gels). Suitable forms of the preparations according to the invention are e.g. antiperspirant/deodorant sprays, antiperspirant/deodorant lotions, antiperspirant/deodorant roll-ons, antiperspirant/deodorant gels, antiperspirant/deodorant creams or antiperspirant/deodorant sticks.

A further subject matter relates to the use of Polyquaternium 37 for producing ethylene oxide free cosmetic or pharmaceutical preparations, in particular of cosmetic or pharmaceutical emulsions.

A further subject matter of the invention relates to the use of Polyquaternium 37 for producing cosmetic or pharmaceutical preparations which comprise less than 1% by weight, in particular less than 0.5% by weight, preferably less than 0.1% by weight, of an interface-active substance. Polyquaternium 37 is particularly suitable for producing cosmetic or pharmaceutical preparations which comprise no interface-active substances.

A further subject matter of the invention relates to the use of Polyquaternium 37 for producing cosmetic or pharmaceutical preparations which comprise a lipophilic phase.

A further subject matter of the invention relates to the use of Polyquaternium 37 for producing cosmetic or pharmaceutical preparations which comprise a lipophilic phase and which comprise less than 1% by weight, in particular less than 0.5% by weight, preferably less than 0.1% by weight, of an interface-active substance.

A further subject matter of the invention relates to the use of Polyquaternium 37 for producing ethylene oxide free preparations selected from the group consisting of antiperspirant/deodorant sprays, antiperspirant/deodorant lotions, antiperspirant/deodorant roll-ons, antiperspirant/deodorant gels, antiperspirant/deodorant creams or antiperspirant/deodorant sticks.

### Examples

The examples below are intended to illustrate the present invention in more detail. All data are in % by weight based on the cosmetic preparation.

### Preparation of the Antiperspirant/Deodorant Preparations:

The swelling in the water phase with Polyquaternium 37 took place at room temperature. Meanwhile, the lipophilic phase—depending on the composition—was heated to 60° C. and then added to the Polyquaternium 37 gel with stirring. The antiperspirant/deodorant active ingredient was added to the system at a temperature of less than 45° C. The formulation was not preserved and the pH was between 3.00 and 4.50.

### Evaluation of the Antiperspirant/Deodorant Formulations:

The formulations were stored for 4 weeks at room temperature (RT) and 40° C.

The assessment of the formulations was carried out as follows:

1=stable
2=phase-stable, although the macroscopic image is inhomogeneous (no oil or water separation)
3=slight water or oil separation
4=unstable (considerably visible water and oil separation)

### Sensory Assessment of the EO Free Antiperspirant/Deodorant Formulations:

The particular formulation was applied to the forearm in a defined amount and rubbed in evenly until it starts to soak in. It takes ca. 3-5 minutes to soak in completely and then the skin feel is determined. A panel consisting of 12 experts carried out the sensory assessment. Questions were asked on the following 6 criteria based on the end feel on the skin:

Spreadability (1=very good; 7=very poor)
Stickiness (1=very slight; 7=very considerable)
Waxy (1=very slight; 7=harsh)
Smoothness (1=high smoothness feel; 7=harsh)
Softness (1=high softness feel; 7=rough)
Care (1=high care feel; 7=uncared for)

TABLE 1
Composition and evaluation of
antiperspirant/deodorant preparations according to the
invention

| | Exam- | Exam- | Exam- | | |
|---|---|---|---|---|---|
| INCI | ple 1 | ple 2 | ple 3 | Example 4 | Example 5 |
| Dist. water | 55.00% | 58.50% | 58.50% | 59.50% | 59.00% |
| Aluminum | 30.00% | 30.00% | 30.00% | 30.00% | 30.00% |
| chlorohydrate | | | | | |
| (50%) | | | | | |
| Pentaerthrityl | 3.00% | 3.00% | — | — | 3.50% |
| distearate | | | | | |
| Cetyl palmitate | — | — | 3.00% | — | — |
| Butyrospermum | — | — | — | 3.00% | — |
| Parkii | | | | | |
| (shea butter) | | | | | |
| Octyldodecanol | 7.00% | 4.00% | 4.00% | 4.00% | 4.00% |
| Polyquaternium 37 | 5.00% | 4.50% | 4.50% | 3.50% | 3.50% |

Evaluation of the stability

| | | | | | |
|---|---|---|---|---|---|
| 1 week/RT | 1 | 1 | 1 | 1 | 1 |
| 1 week/40° C. | 1 | 1 | 1 | 1 | 1 |
| 2 weeks/RT | 1 | 1 | 1 | 1 | 1 |
| 2 weeks/40° C. | 1 | 1 | 1 | 1 | 1 |
| 4 weeks/RT | 1 | 1 | 1 | 1 | 1 |
| 4 weeks/40° C. | 1 | 1 | 1 | 1 | 1 |

Evaluation of the sensory properties

| | | | | | |
|---|---|---|---|---|---|
| Spreadability | 3 | 2 | 5 | 3 | 3 |
| Stickiness | 2 | 3 | 3 | 1 | 2 |
| Waxy | 1 | 2 | 5 | 1 | 1 |
| Smoothness | 3 | 3 | 2 | 1 | 1 |
| Softness | 3 | 2 | 3 | 2 | 3 |
| Care | 2 | 2 | 2 | 3 | 3 |

Examples 1 to 5 according to the invention exhibit very good stability and advantageous sensory properties (low stickiness, high softness, etc.).

TABLE 2
Composition and evaluation of an
antiperspirant/deodorant preparations according to the
prior art and of formulations according to the
invention containing Polyquaternium 37

| | Comparative | | |
|---|---|---|---|
| INCI | example 1 | Example 6 | Example 7 |
| Dist. water | 69.00% | 68.50% | 72.00 |
| Aluminum | 25.00% | 25.00% | 25.00 |
| chlorohydrate (50%) | | | |
| Cetearyl alchol | 1.00% | 1.00% | — |
| Sodium stearoyl | 0.65% | — | — |
| glutamate | | | |
| Propylheptyl | 1.00% | 1.00% | — |
| caprylate | | | |
| Dicaprylyl carbonate | 1.00% | 1.00% | — |
| Dimethicone | 0.50% | 0.50% | — |
| Hydroxyethylcellulose | 0.50% | — | — |
| Glyceryl stearate | 1.35% | — | — |
| citrate | | | |
| Polyquaternium 37 | — | 3.00% | 3.00% |

Evaluation of the stability

| | | | |
|---|---|---|---|
| 1 week/RT | 1 | 1 | 1 |
| 1 week/40° C. | 1 | 1 | 1 |
| 2 weeks/RT | 1 | 1 | 1 |
| 2 weeks/40° C. | 4 | 1 | 1 |
| 4 weeks/RT | 5 | 1 | 1 |
| 4 weeks/40° C. | 5 | 1 | 1 |

Evaluation of the sensory properties

| | | | |
|---|---|---|---|
| Spreadability | 4 | 2 | 3 |
| Stickiness | 6 | 3 | 4 |
| Waxy | 5 | 2 | 1 |
| Smoothness | 5 | 2 | 2 |
| Softness | 5 | 3 | 4 |
| Care | 5 | 2 | 3 |

The comparative example chosen was an antiperspirant/deodorant preparation which comprises no Polyquaternium 37, but ethylene oxide free emulsifier (sodium stearoyl glutamate and glycerol stearate citrate). It was found that the inventive Polyquaternium 37 containing antiperspirant/deodorant preparations of examples 1 to 5 without an emulsifier were not only more stable than the comparison formulation, but have improved sensory properties at the same time. The antiperspirant/deodorant effect was retained, and the ecological concerns relating to ethylene oxide were able to be avoided. Examples 6 and according to the invention likewise exhibited improved stability compared with the prior art (comparative example 1), as well as a reduced stickiness and a high care feel.

Furthermore, antiperspirant/deodorant formulations according to the invention were prepared and compared with antiperspirant/deodorant formulations which comprise ethylene oxide containing substances: the comparison used was a mixture of Polyquaternium 37 (ca. 50% polymer fraction) with diesters of propylene glycol with a mixture of caprylic acid and capric acid (INCI name: propylene glycol dicaprylate/dicaprate) and tridecylpolyoxypropylene polyoxyethylene ether (INCI name: PPG-1-trideceth-6). This mixture is commercially available for example under the trade names Salcare SC 96 or Rheocare CHT E. As can be seen in table 3, only the preparations according to the invention have the viscosities required for the formulation as antiperspirant/deodorant.

TABLE 3

| | Comparative | | Comparative |
|---|---|---|---|
| INCI | example 2 | Example 6 | example 3 |
| Dist. water | 68.5% | 68.50% | 65.50% |
| Aluminum chlorohydrate | 25.00% | 25.00% | 25.00 |
| (50%) | | | |
| Cetearyl alcohol | 1.00% | 1.00% | 1.00% |
| Propylheptyl caprylate | 1.00% | 1.00% | 1.00% |
| Dicaprylyl carbonate | 1.00% | 1.00% | 1.00% |
| Dimethicone | 0.50% | 0.50% | 0.50% |
| Polyquaternium 37, | 3.00% | | 6.00% |
| propylene glycol, | | | |
| dicaprylate/dicaprate, | | | |
| PPG-1 trideceth-6* | | | |
| Polyquaternium 37 | — | 3.00% | |
| Stability at −5° C., RT, | Unstable | Stable for 8 | Stable for 8 |
| 40° C., 45° C. & 50° C. | after one | weeks at all | weeks at all |
| | week at all | temperatures | temperatures |
| | temperatures | | |
| Viscosity (Brookfield; | 800 mPas | 3200 mPas | 22 000 mPas |
| RVT; 23° C.; spindle 5; | | | |
| 10 rpm) | | | |

*ca. 50% fraction Polyquaternium 37

Further formulation examples (R1 to R11) according to the invention are listed below:

| Component | R1 | R2 | R3 | R4 | R5 | R6 | R7 | R8 | R9 | R10 | R11 |
|---|---|---|---|---|---|---|---|---|---|---|---|
| C—Cream, | C | C | C | L | C | L | L | C | L | C | C |
| L—Lotion, | | | | | | | | | | | |
| F—Fluid, | | | | | | | | | | | |
| Trade name | | | | | | | | | | | |
| (INCI) | | | | | | | | | | | |
| Cutina ® PES | | 3 | 3 | 2 | 2 | 3 | 2 | | 2 | | 3 |
| (Pentaery- | | | | | | | | | | | |
| thrityl | | | | | | | | | | | |
| Distearate | | | | | | | | | | | |
| Cutina ® MD | 2 | | | 2 | 3 | 1 | 1 | 3 | 2 | 3 | 1 |
| (Glyceryl | | | | | | | | | | | |
| Stearate) | | | | | | | | | | | |
| Lanette ® 14 | | | 2 | | | | | 2 | | 2.5 | 1 |
| (Myristyl | | | | | | | | | | | |
| Alcohol) | | | | | | | | | | | |
| Lanette ® O | 1 | 2 | | 1 | 2 | 1 | 2.5 | | 1.5 | | |
| (Cetearyl | | | | | | | | | | | |
| Alcohol) | | | | | | | | | | | |
| Novata ® AB | | 1 | 3 | | | | 2 | | | | 2.5 |
| (Coco- | | | | | | | | | | | |
| glycerides | | | | | | | | | | | |
| Cetiol MM ® | 3 | | | 2 | 2.5 | 3 | | 1 | 4 | | |
| (Myristyl | | | | | | | | | | | |
| Myristate) | | | | | | | | | | | |
| Cutina ® CP | | 3 | | | 1.5 | | 2 | 1 | | | 3 |
| (Cetyl | | | | | | | | | | | |
| palmitate | | | | | | | | | | | |
| Cosmedia ® DC | | | 2 | | | | | | | 2.5 | |
| (Hydrogenated | | | | | | | | | | | |
| Dimer | | | | | | | | | | | |
| Dilinoleyl | | | | | | | | | | | |
| Dimethyl- | | | | | | | | | | | |
| carbonate | | | | | | | | | | | |
| Copolymer) | | | | | | | | | | | |
| Cetiol ® SB 45 | 1 | | | | | 1 | | | 1 | | |
| (Shea Butter | | | | | | | | | | | |
| Butyrospermum | | | | | | | | | | | |
| Parkii) | | | | | | | | | | | |
| Myritol ® 318 | | | 3 | | | 4 | | 5 | | | 2 |
| (Caprylic/ | | | | | | | | | | | |
| Capric Tri- | | | | | | | | | | | |
| glyceride) | | | | | | | | | | | |
| Myritol ® PC | | | | 3 | | | 2 | | | 2 | |
| (Propylene | | | | | | | | | | | |
| Glycol | | | | | | | | | | | |
| Dicaprylate/ | | | | | | | | | | | |
| Dicaprate) | | | | | | | | | | | |
| Myritol ® 331 | 4 | 3 | | | | | | 3 | | | |
| (Coco- | | | | | | | | | | | |
| glycerides) | | | | | | | | | | | |
| Finsolv ® TN | | | | | 3 | 4 | | | 2 | | |
| (C12/15 Alkyl | | | | | | | | | | | |
| Benzoate) | | | | | | | | | | | |
| Cetiol ® CC | 2 | | | | 3 | | | 4 | | | |
| (Dicaprylyl | | | | | | | | | | | |
| Carbonate) | | | | | | | | | | | |
| Cetiol ® OE | 3 | 4 | | | | 3 | | | 2 | | 5 |
| (Dicaprylyl | | | | | | | | | | | |
| Ether) | | | | | | | | | | | |
| Dow Corning | | | | 1 | | | | | | | |
| DC ® 245 | | | | | | | | | | | |
| (Cyclopenta- | | | | | | | | | | | |
| siloxane) | | | | | | | | | | | |
| Dow Corning ® | | | | | 1 | | | | | | 1 |
| 2502 | | | | | | | | | | | |
| (Cetyl | | | | | | | | | | | |
| Dimethicone) | | | | | | | | | | | |
| Prisorine ® | | | 2 | | | | 4 | | | 2 | |
| 3758 (Hydrogenated | | | | | | | | | | | |
| Polyiso- | | | | | | | | | | | |
| butene) | | | | | | | | | | | |
| Silikonol | | 0.5 | | | 1 | | | 0.5 | | | |
| Wacker AK ® | | | | | | | | | | | |
| 350 (Di- | | | | | | | | | | | |
| methicone) | | | | | | | | | | | |
| Cetiol ® 868 | 3 | | | | 2 | | | 5 | | | |
| (Ethylhexyl | | | | | | | | | | | |
| Stearate) | | | | | | | | | | | |
| Cetiol ® J 600 | | | | 4 | | 2 | | | 3 | 3 | 2 |
| (Oleyl | | | | | | | | | | | |
| Erucate) | | | | | | | | | | | |
| Ceraphyl ® 45 | | | | | | 3 | | | | | |
| (Diethylhexyl | | | | | | | | | | | |
| Malate) | | | | | | | | | | | |
| Mineral oil | | 3 | | 2 | | | | | | 3 | |
| Cetiol ® | 2 | | | | 3 | 2 | | | 2 | | |
| Sensoft | | | | | | | | | | | |
| (Propylheptyl | | | | | | | | | | | |
| Caprylate) | | | | | | | | | | | |
| Cetiol ® SN | | | 4 | | | | | 3 | | | |
| (Cetearyl | | | | | | | | | | | |
| Isononanoate) | | | | | | | | | | | |
| Cetiol ® B | | 3 | | | | | 2 | | | 3 | |
| (Dibutyl | | | | | | | | | | | |
| Adipate) | | | | | | | | | | | |
| Eutanol ® G | | | | | 5 | | 4 | | 3 | | |
| (Octyl- | | | | | | | | | | | |
| dodecanol) | | | | | | | | | | | |
| Cetiol ® PGL | | | | 3 | | 1 | | | | | 4 |
| (Hexyl- | | | | | | | | | | | |
| decanol, | | | | | | | | | | | |
| Hexyldecyl | | | | | | | | | | | |
| Laurate) | | | | | | | | | | | |
| Dow Corning | | | 1 | | | | | 2 | | 3 | |
| 200—2 cst | | | | | | | | | | | |
| (Dimethicone) | | | | | | | | | | | |
| SFE ® 839 | | | | | 3 | | | | 2 | | |
| (Cyclopenta- | | | | | | | | | | | |
| siloxane and | | | | | | | | | | | |
| Dimethicone/ | | | | | | | | | | | |
| Vinyl | | | | | | | | | | | |
| Dimethicone | | | | | | | | | | | |
| Crosspolymer) | | | | | | | | | | | |
| Almond Oil | | 2 | | | 2.5 | | | 3 | | 2 | |
| (Prunus | | | | | | | | | | | |
| Amygdalus | | | | | | | | | | | |
| Dulcis (Sweet | | | | | | | | | | | |
| Almond) Oil | | | | | | | | | | | |
| Talc | | | 0.5 | | | | | | 1 | | |
| Dry ® Flo Plus | 1 | | | | | 0.5 | | | | | |
| (Aluminium | | | | | | | | | | | |
| Starch | | | | | | | | | | | |
| Octenyl- | | | | | | | | | | | |
| succinate) | | | | | | | | | | | |
| Insect | | | 2 | | | | | | 1 | | 2 |
| Repellent ® | | | | | | | | | | | |
| 3535 (Ethyl | | | | | | | | | | | |
| Butylacetyl- | | | | | | | | | | | |
| amino- | | | | | | | | | | | |
| propionate) | | | | | | | | | | | |
| N,N-Diethyl- | | 2 | | | | | | 2 | | | |
| m-toluamide | | | | | | | | | | | |
| Locron ® L | | | | 30 | | | 30 | 30 | | | 30 |
| (Aluminium | | | | | | | | | | | |
| Chlorhydrate) | | | | | | | | | | | |
| Rezal ® | 15 | | | | 15 | 15 | | | | 15 | |
| 67 = Summit ® | | | | | | | | | | | |
| AZ-7373 | | | | | | | | | | | |
| Powder | | | | | | | | | | | |
| (Aluminum | | | | | | | | | | | |
| Zirconium | | | | | | | | | | | |
| Pentachloro- | | | | | | | | | | | |
| hydrate) | | | | | | | | | | | |
| Rezal ® 36 | | 30 | 30 | | | | | | 30 | | |
| GC = Summit ® | | | | | | | | | | | |
| AZG-442 | | | | | | | | | | | |
| (Aluminum | | | | | | | | | | | |
| Zirconium | | | | | | | | | | | |
| Tetra- | | | | | | | | | | | |
| chlorohydrex | | | | | | | | | | | |
| Gly.) | | | | | | | | | | | |
| Hydagen ® | | | | 2 | | | | | | 2 | |
| C.A.T. | | | | | | | | | | | |
| (Triethyl | | | | | | | | | | | |
| Citrate) | | | | | | | | | | | |
| Irgasan | 2 | | | | 3 | | | | | 1 | |
| (Triclosan) | | | | | | | | | | | |
| Ultragel ® 300 | 3.5 | 5 | 4.5 | 3 | 3.5 | 2.5 | 2 | 4 | 3 | 4 | 3.5 |
| (Polyquaternium | | | | | | | | | | | |
| 37) | | | | | | | | | | | |
| Ethanol | | 3 | | | | 2 | | | | | 3 |
| Butylene | 2 | | | | | | 3 | | | 1 | |
| glycol | | | | | | | | | | | |
| Glycerol | 3 | | | 5 | | | | 7 | | | 4 |
| Water, | q.s. | q.s. | q.s. | q.s. | q.s. | q.s. | q.s. | q.s. | q.s. | q.s. | q.s. |
| preservative, | | | | | | | | | | | |
| etc. | | | | | | | | | | | |

### Antiperspirant/Deodorant Preparation According to the Invention

| Component trade | | |
|---|---|---|
| name | INCI name | % by wt. |
| Water | | 64.0 |
| Cosmedia ® Triple C* | Polyquaternium | 6.0 |
| | 37; dicaprylyl | |
| | carbonate, | |
| | lauryl glucoside | |
| Glycerol | Glycerin | 5.0 |
| Locron ® (50%) | Aluminum | 25.0 |
| | Chlorohydrate | |

*Cosmedia ® Triple C (Cognis GmbH) 50% by weight of Polyquaternium-37; 2% by weight of lauryl glucoside, 48% by weight of dicaprylyl carbonate

1. An ethylene oxide free antiperspirant/deodorant preparation comprising:
a. at least one antiperspirant/deodorant active ingredient, and
b. at least one quaternary polymer with the INCI name Polyquaternium 37.

2. The preparation of claim 1, further comprising:
a lipophilic phase, wherein the lipophilic phase is formed from a liquid oil a wax, or both.

3. The preparation of claim 2, comprising, by weight based on the total weight of the preparation:
a. 1-30% of the at least one antiperspirant/deodorant active ingredient,
b. 0.1-10% of the at least one quaternary polymer with the INCI name Polyquaternium 37, and
c. 1-20% of the lipophilic phase.

4. The preparation of claim 2, comprising, by weight based on the total weight of the preparation:
a. 5-20%, of the at least one antiperspirant/deodorant active ingredient,
b. 1-5% of the at least one quaternary polymer with the INCI name Polyquaternium 37, and
c. 5-15% of the lipophilic phase.

5. The preparation of claim 1, wherein the preparation is in the form of gels or gel creams.

6. The preparation of claim 1, wherein the preparation is in the form of antiperspirant/deodorant sprays, antiperspirant/deodorant lotions, antiperspirant/deodorant roll-ons, antiperspirant/deodorant gels, antiperspirant/deodorant creams or antiperspirant/deodorant sticks.

7. A method for producing ethylene oxide free cosmetic or pharmaceutical preparations comprising using Polyquaternium 37 in the preparation.

8. The method of claim 7, wherein the cosmetic or pharmaceutical preparations comprise no interface-active compounds.

9. The method of claim 7, wherein the cosmetic or pharmaceutical preparations comprise a lipophilic phase.

10. The method of claim 7, wherein the cosmetic or pharmaceutical preparations comprise no interface-active compounds and a lipophilic phase.

11. The method of claim 7, wherein the ethylene oxide free preparations are selected from the group consisting of antiperspirant/deodorant sprays, antiperspirant/deodorant lotions, antiperspirant/deodorant roll-ons, antiperspirant/deodorant gels, antiperspirant/deodorant creams or antiperspirant/deodorant sticks.

12. The preparation of claim 2, comprising, by weight based on the total weight of the preparation:
a. 10-15% of the at least one antiperspirant/deodorant active ingredient,
b. 1-5% of the at least one quaternary polymer with the INCI name Polyquaternium 37, and
c. 5-15% of the lipophilic phase.

13. The preparation of claim 2, wherein the preparation is in the form of gels or gel creams.

14. The preparation of claim 2, wherein the preparation is in the form of antiperspirant/deodorant sprays, antiperspirant/deodorant lotions, antiperspirant/deodorant roll-ons, antiperspirant/deodorant gels, antiperspirant/deodorant creams or antiperspirant/deodorant sticks.

15. The preparation of claim 3, wherein the preparation is in the form of gels or gel creams.

16. The preparation of claim 4, wherein the preparation is in the form of gels or gel creams.

17. The preparation of claim 12, wherein the preparation is in the form of gels or gel creams.

18. The preparation of claim 3, wherein the preparation is in the form of antiperspirant/deodorant sprays, antiperspirant/deodorant lotions, antiperspirant/deodorant roll-ons, antiperspirant/deodorant gels, antiperspirant/deodorant creams or antiperspirant/deodorant sticks.

19. The preparation of claim 4, wherein the preparation is in the form of antiperspirant/deodorant sprays, antiperspirant/deodorant lotions, antiperspirant/deodorant roll-ons, antiperspirant/deodorant gels, antiperspirant/deodorant creams or antiperspirant/deodorant sticks.

20. The preparation of claim 12, wherein the preparation is in the form of antiperspirant/deodorant sprays, antiperspirant/deodorant lotions, antiperspirant/deodorant roll-ons, antiperspirant/deodorant gels, antiperspirant/deodorant creams or antiperspirant/deodorant sticks.
